(19) United States
(12) Patent Application Publication
(10) Pub. No.: US 2025/0295382 A1
(43) Pub. Date: Sep. 25, 2025

(54) ULTRASOUND DIAGNOSTIC APPARATUS

(71) Applicant: FUJIFILM Corporation, Tokyo (JP)

(72) Inventor: Yuta TOKITA, Tokyo (JP)

(73) Assignee: FUJIFILM Corporation, Tokyo (JP)

(21) Appl. No.: 19/079,446

(22) Filed: Mar. 13, 2025

(30) Foreign Application Priority Data
Mar. 25, 2024 (JP) 2024-047952

(51) Int. Cl. A61B 8/00

(52) U.S. Cl. CPC A61B 8/44; A61B 8/461

(57) ABSTRACT

Provided is an ultrasound diagnostic apparatus including a monitor supported by an articulated arm, in which a mechanism that locks the articulated arm and the monitor is simplified. A lock mechanism includes a lock lever that is retracted into a first link of an articulated arm in a normal state and that advances and stands up in a case of locking. An arm base engagement pin of the lock lever and an arm base engagement notch of a base stub are engaged with each other to lock pivoting of the first link with respect to an arm base. An upper end of the lock lever is engaged with a lever engagement part of a lever receiving portion, and a monitor engagement pin is inserted into a monitor engagement hole to lock tilting, up-down movement, and pivoting of a monitor.

### CROSS-REFERENCE TO RELATED APPLICATION

This application claims the priority benefit of Japan application serial no. 2024-047952, filed on Mar. 25, 2024. The entirety of the above-mentioned patent application is hereby incorporated by reference herein and made a part of this specification.

### BACKGROUND OF THE INVENTION

### 1. Field of the Invention

The present invention relates to an ultrasound diagnostic apparatus, and particularly relates to a lock mechanism that locks movement of a monitor that displays an ultrasound image and an articulated arm that supports the monitor.

### 2. Description of the Related Art

An ultrasound diagnostic apparatus used in a medical field generally comprises an apparatus main body, an ultrasound probe that transmits and receives ultrasonic waves to and from a patient, and a monitor that displays an ultrasound image. The monitor is supported by an articulated arm of which one end is supported by the apparatus main body. The monitor is supported by the articulated arm, so that a user who is a doctor or a medical technician can position the monitor in a desired position and orientation. JP 2017-148137A discloses an ultrasound diagnostic apparatus (10) including a monitor (16) supported by an articulated arm (arm mechanism 14). In addition, a lock mechanism that restricts movement of the monitor (16) or the articulated arm (14) in a case in which the ultrasound diagnostic apparatus (10) is moved is shown. The member names and reference numerals in parentheses described above are used in JP 2017-148137A, and are not related to the member names and reference numerals used in the description of the embodiments of the present application.

### SUMMARY OF THE INVENTION

In an ultrasound diagnostic apparatus including a monitor supported by an articulated arm, it is desirable to lock movement of the articulated arm and movement of the monitor so that the monitor does not move unexpectedly when the ultrasound diagnostic apparatus is moved.

The present invention provides a lock mechanism having a simple configuration of locking movement of a monitor and movement of an articulated arm that supports the monitor.

An ultrasound diagnostic apparatus according to an aspect of the present invention comprises a monitor that displays an ultrasound image, an arm base that is provided on an apparatus main body, an articulated arm that has one end supported by the arm base and the other end supporting the monitor, and a lock mechanism that locks movement of the monitor and movement of the articulated arm. The articulated arm includes a monitor base that supports the monitor in a tiltable manner, a first link that has one end pivotally supported by the arm base, and a second link that has one end pivotally supported by the other end of the first link and the other end pivotally supporting the monitor base, and that supports the monitor base to be movable up and down. The lock mechanism includes a lock lever that is mounted on the first link and that is rotationally movable between an advanced position and a retracted position, a first engagement mechanism that locks the pivoting of the first link by engaging the lock lever with the arm base in a case in which the lock lever is in the advanced position, and a second engagement mechanism that locks the tilting of the monitor, the pivoting and up-down movement of the monitor together with the monitor base, and the pivoting of the second link by engaging the lock lever with the monitor in a case in which the lock lever is in the advanced position.

The movement of the articulated arm and the movement of the monitor can be locked by one lock lever, and the lock mechanism can have a simple configuration.

The second engagement mechanism may include a lever receiving portion that is provided at a lower edge portion of the monitor to receive one end of the lock lever in the advanced position. The pivoting of the monitor and the pivoting of the second link may be locked by the lock lever restricting movement of the lever receiving portion in a lock lever transverse direction.

The second engagement mechanism may further include a monitor engagement pin that is provided on one of the lock lever and the lever receiving portion, and a monitor engagement recess portion that is provided on the other of the lock lever and the lever receiving portion and that receives the monitor engagement pin in a case in which the lock lever is in the advanced position. The up-down movement of the monitor may be locked by engagement between the monitor engagement pin and the monitor engagement recess portion. In addition, the tilting of the monitor may be locked by the engagement between the monitor engagement pin and the monitor engagement recess portion and by restriction of movement of the lever receiving portion in a lock lever longitudinal direction via the lock lever.

A tapered portion that is tapered in a direction in which the lock lever enters the lever receiving portion may be provided on a side of the lever receiving portion that receives the lock lever. Even in a case in which the lock lever and the lever receiving portion are misaligned, the lock lever can be guided to a predetermined position.

The monitor engagement pin and the monitor engagement recess portion may be disposed to be engageable with each other in a case in which the monitor is facing downward. By locking the monitor in a state of facing downward, damage to a display surface of the monitor can be prevented.

The first engagement mechanism may include an arm base engagement pin that is provided on one of the lock lever and the arm base, and an arm base engagement recess portion that is provided on the other of the lock lever and the arm base and that receives the arm base engagement pin in a case in which the lock lever is in the advanced position. The pivoting of the first link may be locked by engagement between the arm base engagement pin and the arm base engagement recess portion.

In a case in which the lock lever is in the retracted position, the lock lever may be retracted into the first link, and, in a case in which the lock lever is in the advanced position, the one end of the lock lever may protrude from the first link and may be received in the lever receiving portion.

The movement of the articulated arm and the movement of the monitor can be locked by one lock lever, and the lock mechanism can have a simple configuration.

### BRIEF DESCRIPTION OF THE DRAWINGS

FIG. 1 is a perspective view showing an entire ultrasound diagnostic apparatus.

FIG. 2 is a side view showing the entire ultrasound diagnostic apparatus.

FIG. 3 is a side view showing an articulated arm and a monitor.

FIGS. 4A to 4C are plan views showing an operation of the articulated arm, particularly an operation about one vertical axis.

FIG. 5 is a plan view showing an operation of the articulated arm, particularly a combined operation about a plurality of vertical axes.

FIG. 6 is a diagram showing an operation of the articulated arm in a case in which the monitor is moved up and down.

FIG. 7 is a diagram showing a tilting operation of the monitor.

FIG. 8 is a diagram showing a state where the articulated arm and the monitor are locked.

FIG. 9 is an enlarged view of a mechanism that locks the articulated arm and the monitor.

FIG. 10 is a diagram showing a state where the monitor is tilted forward to a limit.

FIG. 11 is a diagram showing a bottom portion of the monitor as viewed in a direction of an arrow A in FIG. 10.

FIGS. 12A to 12C are diagrams showing an assembly of a lock lever and associated components.

FIG. 13 is a diagram showing a state where the articulated arm and the monitor are locked by the lock lever.

FIGS. 14A to 14C are explanatory diagrams of an engagement operation of a first engagement mechanism.

FIG. 15 is a diagram showing an engaged state of a second engagement mechanism, and is a diagram particularly showing a horizontal cross section.

FIG. 16 is a diagram showing an engaged state of the second engagement mechanism, and is a diagram particularly showing a vertical cross section.

FIG. 17 is a diagram showing a state before engagement between the lock lever and a lever receiving portion.

### DESCRIPTION OF THE PREFERRED EMBODIMENTS

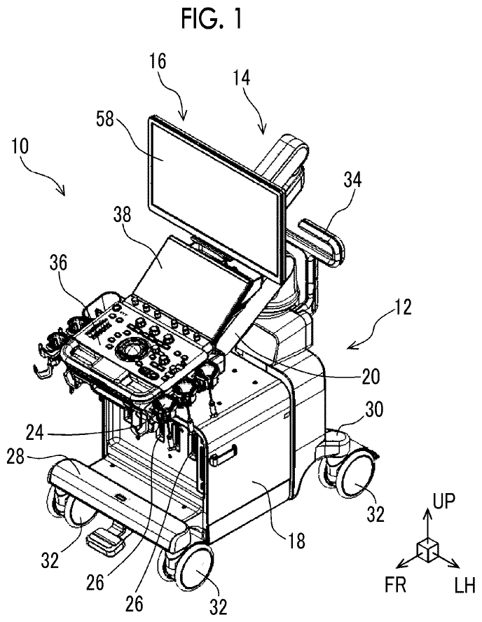

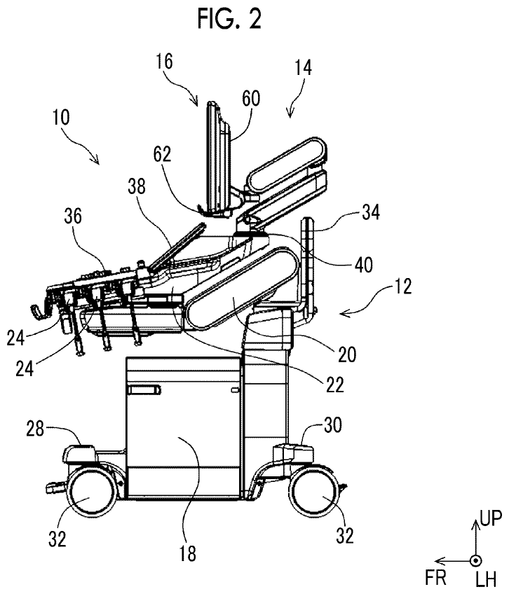

An embodiment according to the present invention will be described with reference to the drawings. FIGS. 1 and 2 are diagrams showing an appearance of an ultrasound diagnostic apparatus 10, FIG. 1 being a perspective view and FIG. 2 being a side view. In FIGS. 1 and 2, a direction indicated by an arrow FR is a forward direction, a direction indicated by an arrow UP is an upward direction, and a direction indicated by an arrow LH is a leftward direction.

The ultrasound diagnostic apparatus 10 includes an apparatus main body 12, an articulated arm 14 supported by the apparatus main body 12, and a monitor 16 that is supported by the articulated arm 14 and that displays an ultrasound image.

The apparatus main body 12 includes a base unit 18 having a substantially box shape, a panel support arm 20 supported by the base unit 18, and an operation panel base 22 supported by the panel support arm 20. The base unit 18 houses an electronic circuit board on which a power supply circuit (not shown), a signal processing circuit that performs signal processing related to transmission and reception of ultrasonic waves, an image processing circuit that forms an ultrasound image, and the like are implemented. A probe connector 26 to which an ultrasound probe 24 that performs transmission and reception of ultrasonic waves is attached and detached is provided on a front surface of the base unit 18. A plurality of the probe connectors 26 may be provided. A front leg part 28 is provided on a lower portion of the front side of the base unit 18, and a rear leg part 30 is provided on a lower portion of a rear side of the base unit 18. Two casters 32 are provided on each of the front leg part 28 and the rear leg part 30, and a total of four casters 32 are disposed corresponding to four corners of the base unit 18. A push handle 34 is provided on the rear surface of the base unit 18 and extends upward.

The panel support arm 20 may support the operation panel base 22 to be movable up and down, and may further support the operation panel base 22 to be pivotable about an vertical axis. An operation panel 36 and a sub monitor 38 are disposed on the operation panel base 22. The operation panel 36 comprises a plurality of switches for operating the ultrasound diagnostic apparatus 10, and an operator such as a pointing device, such as a trackball. The sub monitor 38 may be a liquid crystal display, and displays, for example, a set diagnosis condition. The sub monitor 38 may be a touch panel display. An arm base 40 that supports the articulated arm 14 is provided at a rear end part of the operation panel base 22.

FIGS. 1 and 2 show a state of the ultrasound diagnostic apparatus 10 in which an operator is positioned at the front of the base unit 18, which is a surface on which the probe connector 26 is disposed, and the operation panel 36 and the monitor 16 face the operator. Positions of the articulated arm 14 and the monitor 16 shown in FIGS. 1 and 2 are referred to as “home positions”. At the home position, the articulated arm 14 extends along a front-rear direction in a folded state, and the monitor 16 faces forward. In the following description, terms indicating a positional relationship and a direction, such as front, rear, left, right, up, and down, indicate a positional relationship and a direction in the state shown in FIGS. 1 and 2 unless otherwise specified.

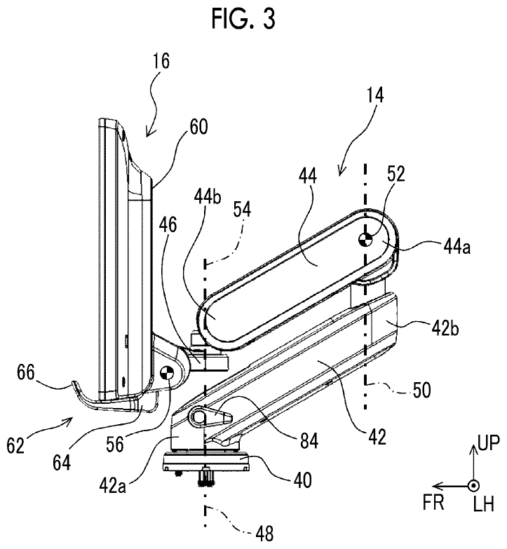

FIG. 3 is an enlarged side view of the articulated arm 14 and the monitor 16 in the home position. The articulated arm 14 includes a first link 42 having one end supported by the arm base 40, a second link 44 having one end supported by the other end of the first link 42, and a monitor base 46 supported by the other end of the second link 44. The monitor base 46 supports the monitor 16. In a path along the articulated arm 14 connected in order of the first link 42, the second link 44, and the monitor base 46, an end part of each element constituting the articulated arm 14, which is close to the arm base 40, is referred to as a proximal end, and an end part on an opposite side is referred to as a distal end.

The first link 42 is supported by the arm base 40 at a proximal end 42a and is disposed to be tilted to extend toward a distal end 42b located at a higher position. The first link 42 is pivotable about a first vertical axis 48 defined to penetrate the arm base 40 along a vertical direction, that is, is rotationally movable in a horizontal plane. In the following description, rotational movement around an axis extending in the vertical direction is referred to as “pivoting”. The second link 44 is supported by the distal end 42b of the first link 42 at a proximal end 44a and is pivotable about a second vertical axis 50 defined to penetrate the distal end 42b along the vertical direction. Therefore, the articulated arm 14 bends and stretches at a portion where the first link 42 and the second link 44 are connected to each other in a plan view. In a case in which the articulated arm 14 is in the home position, the second link 44 is disposed in parallel with the first link 42 as shown in FIG. 3. The second link 44 is rotationally movable about a first horizontal axis 52 defined to penetrate the proximal end 44a along a horizontal direction and along a direction orthogonal to a longitudinal direction of the second link 44. A distal end 44b of the second link 44 is moved in an up-down direction in an arc shape by the rotational movement. The monitor base 46 is supported by the distal end 44b of the second link 44 and is pivotable about a third vertical axis 54 defined to penetrate the distal end 44b along the vertical direction. The monitor 16 changes its orientation in a left-right direction as the monitor base 46 pivots. In addition, the monitor 16 is tilted about a second horizontal axis 56 defined to penetrate the monitor base 46 along the horizontal direction and along a direction parallel to the monitor 16. Through this tilting, the monitor 16 can be directed downward and upward.

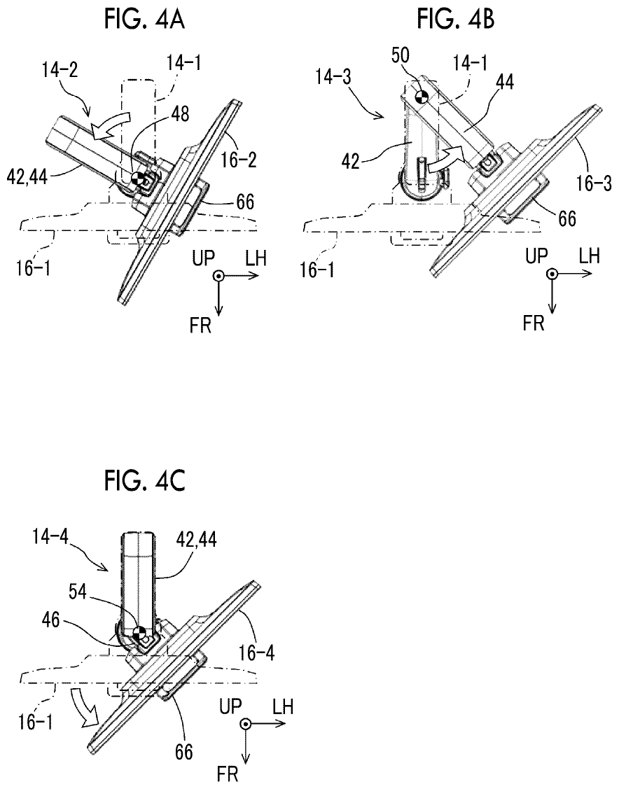

The monitor 16 includes a display unit 58 (see FIG. 1) such as a liquid crystal panel and a monitor housing 60 that holds the display unit 58 such that a display surface of the display unit 58 is exposed and a rear surface of the display unit 58 is covered. A monitor handle 62 is mounted on a lower edge of the monitor housing 60 at the center in the left-right direction. The monitor handle 62 includes a handle base 64 that is fixed to the monitor housing 60, and a grip portion 66 that extends forward from the handle base 64 beyond a front surface of the display unit 58. The grip portion 66 has a U-shape structure in a plan view (see FIGS. 4A to 4C), and the operator grips a portion corresponding to a vertical line of the U-shape structure to move the monitor 16 and to adjust a position and an orientation of the monitor 16.

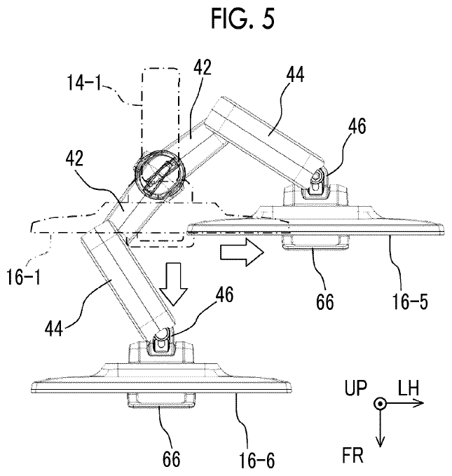

FIGS. 4A to 4C and FIG. 5 are diagrams showing how each element of the articulated arm 14 and the monitor 16 pivot. The articulated arm 14-1 and the monitor 16-1 in the home position are shown by dashed lines.

FIG. 4A shows a state in which the articulated arm 14 pivots about the first vertical axis 48 from the home position so that the monitor 16 faces diagonally leftward. A positional relationship between the elements (the first link 42, the second link 44, and the monitor base 46) of the articulated arm 14-2 in this state and a positional relationship between the articulated arm 14-2 and the monitor 16-2 are the same as those in a case in which the articulated arm 14-1 and the monitor 16-1 are in the home position, and the articulated arm 14 and the monitor 16 as a whole integrally rotate and move about the first vertical axis 48.

FIG. 4B shows a state where the second link 44 pivots about the second vertical axis 50 from the home position so that the monitor 16 faces leftward. In the case of the articulated arm 14-3 in this state, the first link 42 is in the home position, and the first link 42 and the second link 44 are positioned at an angle in a plan view. In addition, a relationship between the second link 44 and the monitor 16-3 in this state is the same as the home position.

FIG. 4C shows a state where the monitor base 46 pivots about the third vertical axis 54 from the home position so that the monitor 16 faces leftward. In the case of the articulated arm 14-4 in this state, the first link 42 and the second link 44 are in the home position, and the first link 42 and the second link 44, and the monitor base 46 are positioned at an angle in a plan view. As a result, the positional relationship between the second link 44 and the monitor 16-4 is changed from the home position.

The articulated arm 14 and the monitor 16 shown in FIGS. 4A, 4B, and 4C are in a state achieved by the pivoting of the articulated arm 14 about only one vertical axis. Meanwhile, FIG. 5 shows a state achieved by an operation of the articulated arm 14 about a plurality of vertical axes. Through the operation about the plurality of vertical axes, the monitor 16 in the home position can be moved in parallel from the home position, with the monitor 16-5 shown moved in parallel to the left side and the monitor 16-6 shown moved in parallel to the front side.

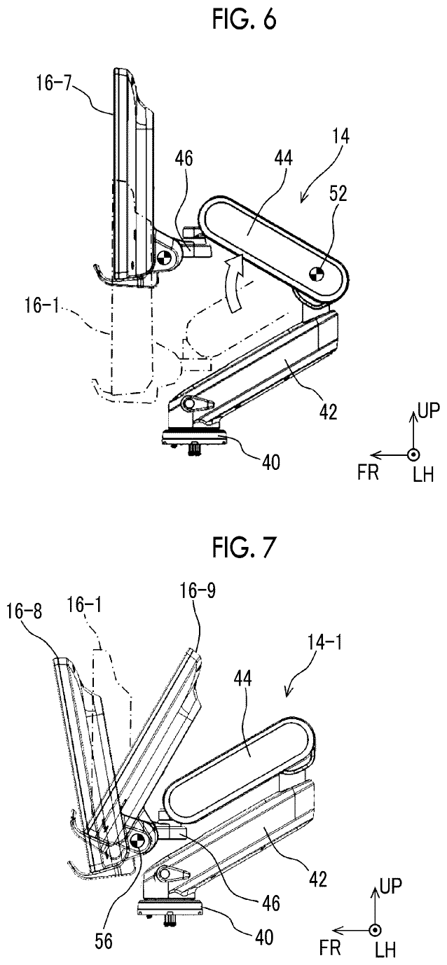

FIG. 6 is a diagram showing movement of the articulated arm 14 in a case in which the monitor 16 is moved up and down. In a case in which the monitor 16-1 in the home position is moved upward, the second link 44 of the articulated arm 14 is rotationally moved about the first horizontal axis 52. The second link 44 has a parallel link structure, so that even in a case in which the second link 44 is rotationally moved, the monitor base 46 moves up and down in parallel while maintaining its attitude. The monitor 16 supported by the monitor base 46 also moves up and down while maintaining its orientation. FIG. 6 shows the monitor 16-7 that is moved in parallel upward.

FIG. 7 is a diagram showing the tilting of the monitor 16. The monitor 16 is tilted about the second horizontal axis 56. FIG. 7 shows the monitor 16-8 that is tilted forward and faces downward and the monitor 16-9 that is tilted backward and faces upward from the position of the monitor 16-1 that faces forward in the horizontal direction. The monitor 16-8 shows a state where a depression angle is 10°, and the monitor 16-9 shows a state where an elevation angle is 30°.

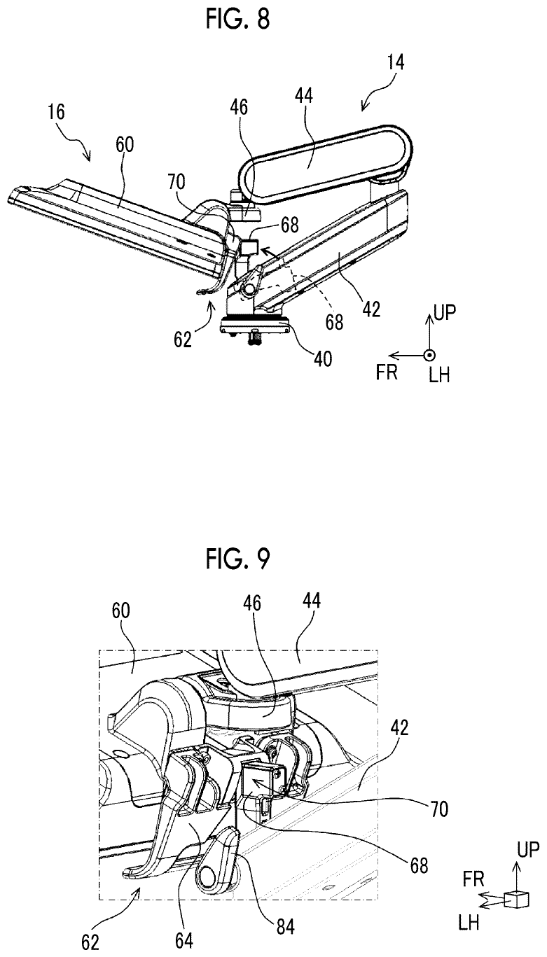

FIGS. 8 and 9 are diagrams showing a state where the movement of the articulated arm 14 and the movement of the monitor 16 are locked. FIG. 8 is a side view, and FIG. 9 is an enlarged view of a mechanism portion related to locking in a case of locking. The positions of the locked articulated arm 14 and monitor 16 will be referred to as “locked positions” hereinafter. In a case of locking, a lock lever 68 that is located in the first link 42 in a normal state is raised, and the lock lever 68 in the raised state is received in a lever receiving portion 70 provided on the monitor 16. In FIG. 8, the lock lever 68 located in the first link 42 is shown by a broken line, and the raised lock lever 68 is shown by a solid line. The lever receiving portion 70 is provided on the handle base 64 of the monitor handle 62 fixed to the monitor housing 60. In the locked position, the monitor 16 is in a state of being tilted forward to a limit restricted by a stopper, and the lever receiving portion 70 provided at a bottom portion of the monitor 16 faces an upper end of the raised lock lever 68 in the substantially front-rear direction.

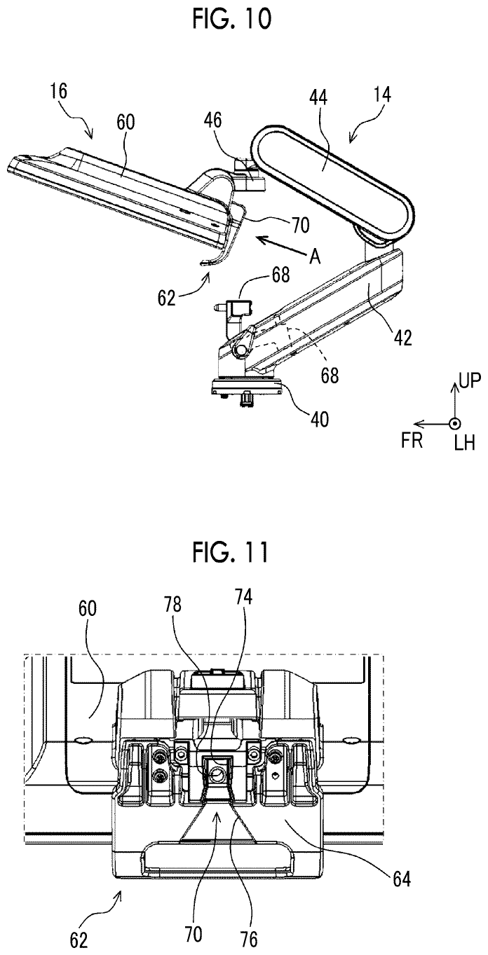

FIG. 10 is a diagram showing the monitor 16 in a state before being locked and being tilted forward to a limit position and the articulated arm 14 at that time. In addition, FIG. 11 shows the bottom portion of the monitor 16 as viewed in a direction of an arrow A shown in FIG. 10. In FIG. 10, the entire upper end of the raised lock lever 68 is shown. The lock lever 68 has, at the upper end thereof, a monitor engagement pin 72 that extends toward the bottom portion of the monitor 16 in the locked position. The lever receiving portion 70 has a groove shape, and has, in an upper portion in FIG. 11, a lever engagement part 74 that engages with the upper end of the lock lever 68 and has, in a lower portion in FIG. 11, a tapered part 76 having a tapered shape that widens in the left-right direction downward. A monitor engagement hole 78, which is an engagement recess portion that receives the monitor engagement pin 72, is formed in a groove bottom surface of the lever engagement part 74.

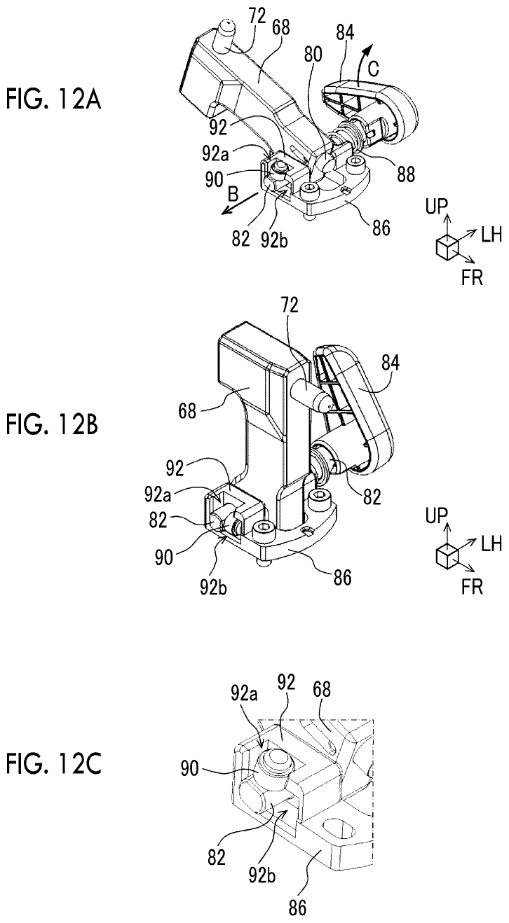

FIGS. 12A to 12C are diagrams showing a detailed structure of the lock lever 68 and components around the lock lever 68. FIG. 12A shows a state where the lock lever 68 is retracted into the first link 42, and FIG. 12B shows a state where the lock lever 68 advances from the first link 42 and stands up to lock the monitor 16 or the like. FIG. 12C shows an enlarged view of the detailed structure. FIGS. 12A to 12C show the lock lever 68 and components around the lock lever 68 in a state where the articulated arm 14 is in the home position. In a state where the lock lever 68 stands up, that is, in the state shown in FIG. 12B, an end on the upper side is referred to as an upper end, and an end on the lower side is referred to as a lower end.

The lock lever 68 has a substantially columnar shape. The monitor engagement pin 72 is provided on an upper end portion of the lock lever 68 so as to be movable forward and backward, and an arm base engagement pin 80 is provided on a lower end portion of the lock lever 68 so as to be movable forward and backward. The monitor engagement pin 72 is movable forward and backward along a direction substantially orthogonal to the longitudinal direction of the lock lever 68, and the arm base engagement pin 80 is movable forward and backward substantially along the longitudinal direction of the lock lever 68. The monitor engagement pin 72 and the arm base engagement pin 80 are biased in an advancing direction by a spring, particularly a coil spring.

A lever shaft 82 passes through the lower end part of the lock lever 68. The lock lever 68 and the lever shaft 82 rotationally move integrally about a central axis of the lever shaft 82, but are connected to be movable relative to each other in the longitudinal direction of the lever shaft 82, that is, in a direction along the central axis. A lever operating handle 84 is fixed to one end of the lock lever 68. The lever operating handle 84 is disposed on a side surface of the first link 42 (see FIGS. 3, 9, and the like). The lever shaft 82 is supported to be rotationally movable by the first link 42, particularly a lever base 86 fixed to an internal frame member of the first link 42, and is also allowed to move along the central axis of the lever shaft 82 within a predetermined range. On the other hand, the movement of the lock lever 68 along the central axis of the lever shaft 82 is restricted by the lever base 86. Further, a coil spring 88 is disposed coaxially with the lever shaft 82. The coil spring 88 biases the lever operating handle 84 and the lever shaft 82 to the left side LH.

A lever restricting pin 90 is provided at an end of the lever shaft 82 opposite to an end where the lever operating handle 84 is provided. The lever restricting pin 90 protrudes in a direction orthogonal to an axis of the lever shaft 82 and moves rotationally integrally with the lever shaft 82 about the central axis of the lever shaft 82. The lever base 86 is provided with a restriction pin receiver 92 that engages with the lever restricting pin 90. The restriction pin receiver 92 is formed with pin receiving portions 92a and 92b for receiving the lever restricting pin 90 at two locations. In a case in which the lever restricting pin 90 is received in the first pin receiving portion 92a, the lock lever 68 is positioned in a retracted position where the lock lever 68 is retracted into the first link 42 and is maintained in the position (see FIG. 12A). In addition, in a case in which the lever restricting pin 90 is received in the second pin receiving portion 92b, the lock lever 68 is positioned at an advanced position where the lock lever 68 stands up and is maintained in the position (see FIG. 12B).

In order to make the lock lever 68 stand up from the retracted position, first, the lever operating handle 84 is pushed against the biasing force of the coil spring 88 to move the lever shaft 82 in a direction of an arrow B. This movement of the lever shaft 82 causes the lever restricting pin 90 to disengage from the first pin receiving portion 92a of the restriction pin receiver 92, allowing the rotational movement about the central axis of the lever shaft 82. Next, in a case in which the lever operating handle 84 is rotationally moved in a direction of an arrow C, the movement of the lever operating handle 84 causes the lever shaft 82 and the lock lever 68 to be rotationally moved. In a case in which the lock lever 68 stands up, a force of pushing the lever operating handle 84 is released. As a result, the lever restricting pin 90 is engaged with the second pin receiving portion 92b, and the lock lever 68 is positioned in the advanced position. Similarly, in a case of moving the lock lever 68 from the advanced position to the retracted position, the lever operating handle 84 is pushed and rotationally moved in a direction opposite to the arrow C, and then the pushing force is released.

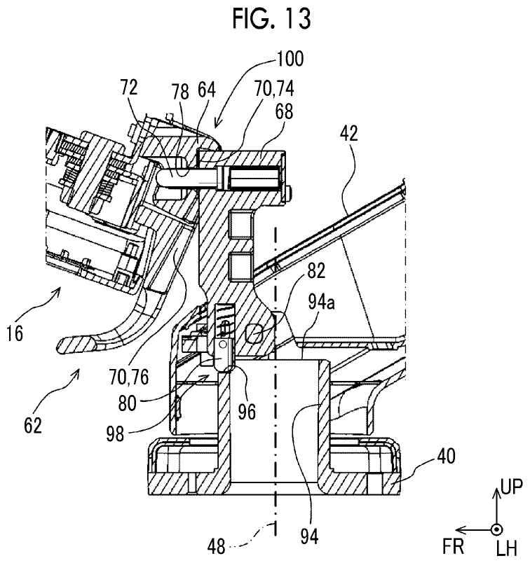

FIG. 13 is a cross-sectional view showing a state where the articulated arm 14 and the monitor 16 are locked by the lock lever 68. The arm base 40 includes a base stub 94 that extends upward. The base stub 94 has a cylindrical shape, and the first link 42 is supported by the base stub 94 via a bearing so as to be rotationally movable. Therefore, a central axis of the base stub 94 is the first vertical axis 48. A notch 96 is formed at one position on a periphery of an annular top surface 94a of the base stub 94. In a case in which the lock lever 68 is in the advanced position, the arm base engagement pin 80 advances from a lower end of the lock lever 68 due to the biasing force of the spring and engages with the notch 96, and the pivoting of the first link 42 about the first vertical axis 48 is locked. The notch 96 is a recess portion that receives the arm base engagement pin 80 in a case in which the lock lever 68 is in the advanced position. Hereinafter, the notch 96 will be referred to as an arm base engagement notch 96. The arm base engagement pin 80 and the arm base engagement notch 96 constitute an engagement mechanism that engages the lock lever 68 and the arm base 40 with each other to lock the pivoting of the first link 42. This engagement mechanism will be referred to as a first engagement mechanism 98.

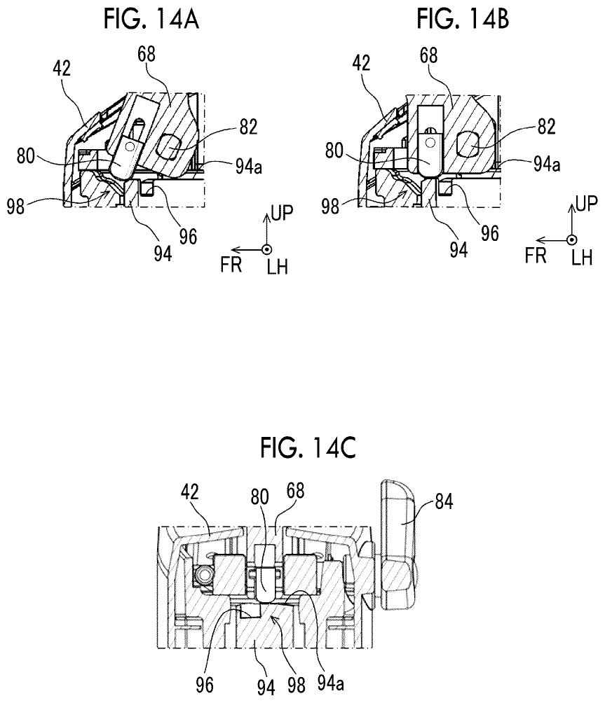

FIGS. 14A to 14C are diagrams for describing a process of locking the pivoting of the first link 42 from a state where the arm base engagement pin 80 and the arm base engagement notch 96 are misaligned in a circumferential direction. FIG. 14A shows a state before the lock lever 68 reaches the advanced position, and FIGS. 14B and 14C show a state where the lock lever 68 reaches the advanced position.

In a case in which the first link 42 is in a position (for example, see FIG. 4A) deviated from the home position by pivoting, and the lock lever 68 is raised, the arm base engagement pin 80 comes into contact with the base stub 94 at a position deviated from the arm base engagement notch 96. The state in this case is shown in FIG. 14A. As the lock lever 68 is rotationally moved, the arm base engagement pin 80 is pushed by the top surface 94a of the base stub 94, and retracts against the biasing force of the spring, riding onto the top surface 94a. The state in this case is shown in FIGS. 14B and 14C. In order for the arm base engagement pin 80 to easily ride onto the top surface 94a of the base stub, a chamfer may be provided at an edge of the top surface 94a of the base stub on an outer peripheral side. In addition, an end surface of the arm base engagement pin 80 may be a curved surface such as a spherical surface. The first link 42 is rotationally moved about the first vertical axis 48 toward the home position in a state where the arm base engagement pin 80 rides onto the top surface 94a of the base stub. As a result, the arm base engagement pin 80 moves on the top surface 94a of the base stub in the circumferential direction. In a case in which the arm base engagement pin 80 is moved to the position of the arm base engagement notch 96, the arm base engagement pin 80 advances into the arm base engagement notch 96 by the biasing force of the spring and engages with the arm base engagement notch 96.

In order to unlock the first link 42, the lock lever 68 is rotationally moved to the retracted position. By the rotational movement of the lock lever 68, the arm base engagement pin 80 is moved radially outward of the base stub 94 and comes out of the arm base engagement notch 96, thereby being released from the engagement therewith.

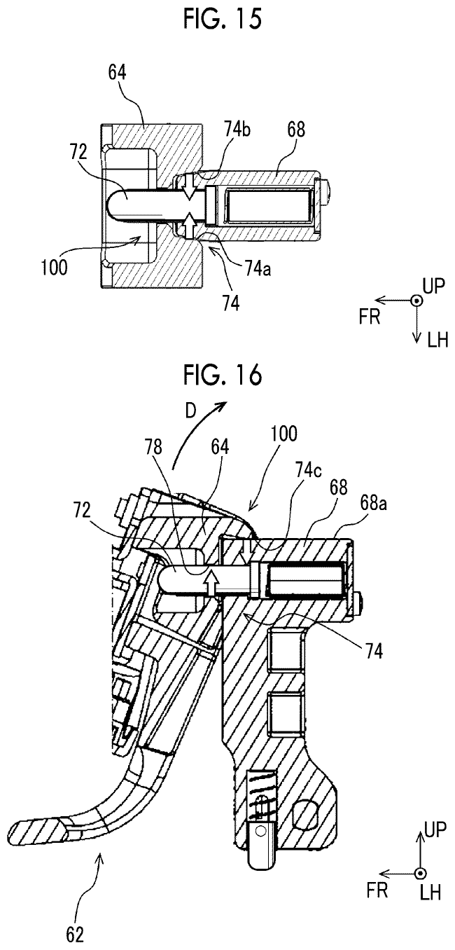

Returning to FIG. 13, the engagement between the monitor 16 and the lock lever 68 will be described. The upper end of the lock lever 68 in the advanced position is engaged with the lever engagement part 74 of the lever receiving portion 70 provided at the bottom portion of the monitor 16, more specifically, at the monitor handle 62. In addition, the monitor engagement pin 72 is biased by a spring and enters the monitor engagement hole 78. By the monitor engagement pin 72 being engaged with the monitor engagement hole 78, the movement of the monitor 16 in the up-down direction and in the left-right direction with respect to the lock lever 68 is restricted. That is, the pivoting of the monitor 16 about the second vertical axis 50, the pivoting of the monitor 16 about the third vertical axis 54, and the rotational movement of the second link 44 about the first horizontal axis 52 are locked. Further, as shown in the horizontal cross section of FIG. 15, left and right side surfaces 74a and 74b of the lever engagement part 74 formed on the handle base 64 come into contact with left and right side surfaces of the upper end of the lock lever 68, thereby restricting the left and right movement of the lever receiving portion 70, that is, the movement in a direction of crossing the lock lever 68. Therefore, the left and right movement of the monitor 16 is restricted. That is, the pivoting of the monitor 16 about the second vertical axis 50 and the pivoting of the monitor 16 about the third vertical axis 54 are locked. Since the lock lever 68 is provided on the first link 42, the monitor 16 is locked against up, down, left, and right movement with respect to the first link 42, and the second link 44 is also locked against pivoting about the second vertical axis 50 and rotational movement about the first horizontal axis 52 with respect to the first link 42. Further, in a case in which the pivoting of the first link 42 is locked by the first engagement mechanism 98, the monitor 16 is locked against up-down movement and pivoting with respect to the apparatus main body 12, and the second link 44 is locked against pivoting about the vertical axis and rotational movement about the horizontal axis with respect to the apparatus main body 12.

The monitor 16, which has been tilted forward to a lower limit, is locked from tilting upward by the engagement of the lock lever 68 and the monitor engagement pin 72 with the lever receiving portion 70, particularly with the lever engagement part 74. As shown in FIG. 16, in a case in which the monitor 16 is tilted upward as in an arrow D, a lower portion of the monitor engagement hole 78 comes into contact with the monitor engagement pin 72, and an upper surface 74c of the lever engagement part 74 comes into contact with an upper end surface 68a of the lock lever 68. As a result, the tilting of the monitor 16 is locked.

The lever receiving portion 70 having the monitor engagement hole 78 and the lever engagement part 74, and the upper end portion of the lock lever 68 comprising the monitor engagement pin 72 constitute an engagement mechanism that locks the tilting of the monitor 16, the pivoting and up-down movement of the monitor 16, and the pivoting and rotational movement of the second link 44. This engagement mechanism will be referred to as a second engagement mechanism 100.

In order to set the second engagement mechanism 100 to the locked state, first, as shown in FIG. 10, the monitor 16 is tilted forward to a limit at a position higher than the locked position. Thereafter, the monitor 16 is lowered downward.

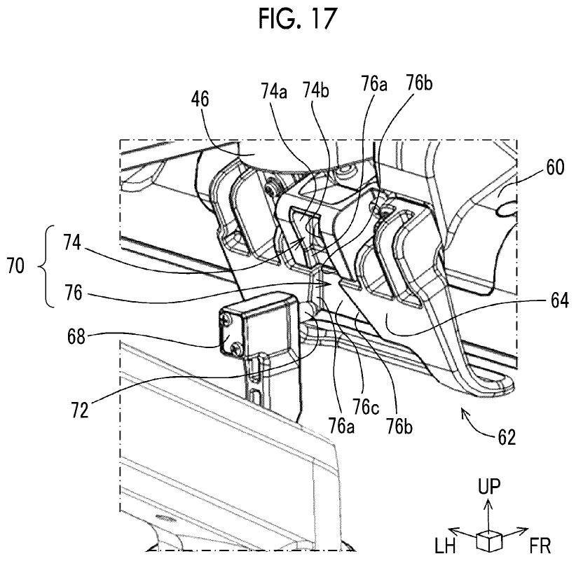

FIG. 17 is a diagram showing a state where the lever receiving portion 70 is close to the lock lever 68 in a process of lowering the monitor 16. The lever receiving portion 70 includes the lever engagement part 74 positioned on the upper side and the tapered part 76 positioned on the lower side in FIG. 17. The lever receiving portion 70 is a groove formed on a bottom surface of the handle base 64, and in FIG. 17, an upper end of the groove is closed and a lower end is open. In a case in which the upper end of the lock lever 68 is engaged with the side surfaces 74a and 74b of the lever engagement part 74, the side surfaces 74a and 74b prevent the lock lever 68 from moving relatively left and right. The tapered part 76 is defined by left and right side walls of the groove, that is, side surfaces 76a and 76b, and a bottom surface 76c which is a bottom of the groove. An interval between the side surfaces 76a and 76b of the tapered part 76 is wide at the bottom and narrow at the top. Each of the side surfaces 76a and 76b of the tapered part 76 is connected to a lower end of the side surfaces 74a and 74b of the lever engagement part at an upper end. The bottom surface 76c of the tapered part 76 is also connected to a lower end of the bottom surface of the lever engagement part at an upper end.

In a case in which the monitor 16 that has been tilted forward is lowered, the lock lever 68 enters the lever receiving portion 70 from below. In a case in which the lock lever 68 enters the lever receiving portion 70, a distal end of the monitor engagement pin 72 comes into contact with the bottom surface 76c of the tapered part of the lever receiving portion 70 and is pushed. As the monitor 16 is lowered, the monitor engagement pin 72 moves toward the retracted position. In a case in which the monitor 16 is further lowered and the monitor engagement pin 72 and the monitor engagement hole 78 are aligned with each other, the monitor engagement pin 72 advances into the monitor engagement hole 78 by the biasing force of the spring, and the monitor engagement pin 72 and the monitor engagement hole 78 are engaged with each other. In a case in which the lock lever 68 and the lever receiving portion 70 are misaligned in the left-right direction, the upper end of the monitor engagement pin 72 or the lock lever 68 comes into contact with the side surfaces 76a and 76b of the tapered part of the lever receiving portion 70. Further, in a case in which the monitor 16 is lowered, the lock lever 68 is guided to the lever engagement part 74 by the side surfaces 76a and 76b.

The upper end of the lock lever 68 is engaged with the lever engagement part 74 of the lever receiving portion 70, and the monitor engagement pin 72 is engaged with the monitor engagement hole 78 formed on the bottom surface of the lever engagement part 74, so that the second engagement mechanism 100 is in the engaged state. As a result, the tilting of the monitor 16 is locked, the pivoting and up-down movement of the monitor 16 together with the monitor base 46 are locked, and the pivoting of the second link 44 with respect to the first link 42 is locked.

In order to set the articulated arm 14 and the monitor 16 to the locked state from the use state of the ultrasound diagnostic apparatus 10, first, the lever operating handle 84 is rotationally moved to raise the lock lever 68 to set the lock lever 68 to the advanced position. In a case in which the arm base engagement pin 80 and the arm base engagement notch 96 are misaligned in the circumferential direction, the first link 42 is pivoted about the first vertical axis 48 to align the arm base engagement pin 80 and the arm base engagement notch 96 with each other and to engage the arm base engagement pin 80 and the arm base engagement notch 96 with each other. As a result, the first link 42 is locked by the first engagement mechanism 98.

Next, the monitor 16 and the second link 44 are locked by the second engagement mechanism 100. First, the monitor 16 is tilted forward to the limit. In this case, the grip portion 66 of the monitor handle 62 is positioned above the upper end of the lock lever 68 in the advanced position. In a case in which the monitor 16 that has been tilted forward is lowered, the upper end of the lock lever 68 and the monitor engagement pin 72 are guided by the tapered part 76 of the lever receiving portion 70, and the lock lever 68 reaches the lever engagement part 74. In addition, the monitor engagement pin 72 is pushed by the bottom surface 76c of the tapered part 76 and retracts, and then the monitor engagement pin 72 advances into the monitor engagement hole 78 in a case in which the monitor engagement pin 72 reaches a position facing the monitor engagement hole 78. As a result, the monitor 16 and the second link 44 are locked by the second engagement mechanism 100. In addition, the second link 44 and the first link 42 are also locked. As a result, the movement of the articulated arm 14 itself and the relative movement of the articulated arm 14 with the monitor 16 are locked, and the articulated arm 14 and the monitor 16 are integrated. The articulated arm 14 and the monitor 16 are fixed to the apparatus main body by the locking with the first engagement mechanism 98 and the second engagement mechanism 100.

The order of the locking with the first engagement mechanism 98 and the locking with the second engagement mechanism 100 may be reversed. That is, the monitor 16 is tilted forward and is lowered, the monitor 16 and the second link 44 are set to the locked state, and then the articulated arm 14 and the monitor 16 are integrally pivoted to engage the arm base engagement pin 80 and the arm base engagement notch 96 with each other, so that the articulated arm 14 and the monitor 16 together with each other are fixed to the apparatus main body.

In the first engagement mechanism 98, the arm base engagement pin 80 and the arm base engagement notch 96 may be disposed to be exchanged. That is, the base stub having the notch may be rotated integrally with the first link 42, and the engagement pin that moves forward and backward with respect to the notch may be provided on the arm base 40. In the second engagement mechanism 100, the monitor engagement pin 72 and the monitor engagement hole 78 may be disposed to be exchanged. That is, the engagement pin that moves forward and backward may be disposed on the bottom surface of the lever receiving portion 70, and the engagement hole that engages with the engagement pin may be disposed at the upper end of the lock lever.

What is claimed is:

1. An ultrasound diagnostic apparatus comprising:
a monitor that displays an ultrasound image;
an arm base that is provided on an apparatus main body;
an articulated arm that has one end supported by the arm base and the other end supporting the monitor; and
a lock mechanism that locks movement of the monitor and movement of the articulated arm,
wherein the articulated arm includes:
a monitor base that supports the monitor in a tiltable manner,
a first link that has one end pivotally supported by the arm base, and
a second link that has one end pivotally supported by the other end of the first link and the other end pivotally supporting the monitor base, and that supports the monitor base to be movable up and down, and
the lock mechanism includes:
a lock lever that is mounted on the first link and that is rotationally movable between an advanced position and a retracted position,
a first engagement mechanism that locks the pivoting of the first link by engaging the lock lever with the arm base in a case in which the lock lever is in the advanced position, and
a second engagement mechanism that locks the tilting of the monitor, the pivoting and up-down movement of the monitor together with the monitor base, and the pivoting of the second link by engaging the lock lever with the monitor in a case in which the lock lever is in the advanced position.

2. The ultrasound diagnostic apparatus according to claim 1,
wherein the second engagement mechanism includes a lever receiving portion that is provided at a lower edge portion of the monitor to receive one end of the lock lever in the advanced position, and
the pivoting of the monitor and the pivoting of the second link are locked by the lock lever restricting movement of the lever receiving portion in a lock lever transverse direction.

3. The ultrasound diagnostic apparatus according to claim 2,
wherein the second engagement mechanism includes a monitor engagement pin that is provided on one of the lock lever and the lever receiving portion, and a monitor engagement recess portion that is provided on the other of the lock lever and the lever receiving portion and that receives the monitor engagement pin in a case in which the lock lever is in the advanced position,
the up-down movement of the monitor is locked by engagement between the monitor engagement pin and the monitor engagement recess portion, and
the tilting of the monitor is locked by the engagement between the monitor engagement pin and the monitor engagement recess portion and by restriction of movement of the lever receiving portion in a lock lever longitudinal direction via the lock lever.

4. The ultrasound diagnostic apparatus according to claim 3,
wherein a tapered portion that is tapered in a direction in which the lock lever enters the lever receiving portion is provided on a side of the lever receiving portion that receives the lock lever.

5. The ultrasound diagnostic apparatus according to claim 3,
wherein the monitor engagement pin and the monitor engagement recess portion are disposed to be engageable with each other in a case in which the monitor is facing downward.

6. The ultrasound diagnostic apparatus according to claim 2,
wherein the first engagement mechanism includes an arm base engagement pin that is provided on one of the lock lever and the arm base, and an arm base engagement recess portion that is provided on the other of the lock lever and the arm base and that receives the arm base engagement pin in a case in which the lock lever is in the advanced position, and
the pivoting of the first link is locked by engagement between the arm base engagement pin and the arm base engagement recess portion.

7. The ultrasound diagnostic apparatus according to claim 6,
wherein, in a case in which the lock lever is in the retracted position, the lock lever is retracted into the first link, and, in a case in which the lock lever is in the advanced position, the one end of the lock lever protrudes from the first link and is received in the lever receiving portion.
